(19) United States
(12) Patent Application Publication
(10) Pub. No.: US 2025/0000717 A1
(43) Pub. Date: Jan. 2, 2025

(54) LEAK-RESISTANT UNDERWEAR

(71) Applicant: Bondville (PVT) Ltd, Batuwita (LK)

(72) Inventors: Tuse Cooray Mohotti Gurunnanselage Janaka Jeewantha COORAY, Rathnapura (LK); Kankani Gamage Gayan YAPA, Attidiya (LK); Sudharini Piumali DIAS, Anamaduwa (LK); Hiniduma Vithanage Pahan PRASANGE, Galle (LK); Roshan Sanjeewa DELWITA, Baddegama (LK); Rathnayaka Mudiyanselage Jayamal Chathuranga Bandara Rathnayaka, Badulla (LK)

(21) Appl. No.: 18/753,351

(22) Filed: Jun. 25, 2024

Related U.S. Application Data
(60) Provisional application No. 63/511,099, filed on Jun. 29, 2023.

(51) Int. Cl. A61F 13/494; A61F 13/496

(52) U.S. Cl. CPC A61F 13/49426; A61F 13/49446; A61F 13/496

(57) ABSTRACT

Leak-resistant underwear is provided. The leak-resistant underwear includes a gusset having a multi-layer construction. The multi-layer construction includes a sealed gusset assembly and a gusset portion of a body fabric. The sealed gusset assembly includes a next-to-skin layer, an absorbent layer, a blocking layer, polymer component. The absorbent layer is disposed between the next-to-skin layer and the blocking layer. The polymer component forms a seal between the next-to-skin layer and the blocking layer along at least a portion of respective perimeters of the next-to-skin layer and the blocking layer. The sealed gusset assembly is configured to be disposed between a wearer's skin and the gusset portion of the body fabric. In some cases, a portion of the perimeter of the gusset is sealed with a sealing tape.

### FIELD

Embodiments relate to leak-resistant and/or leak-proof underwear or panties. For example, an example embodiment relates to leak-resistant and/or leak-proof reusable menstrual underwear.

### BACKGROUND

Reusable menstrual underwear is generally configured to absorb and contain fluid such as menstrual fluid, urine, or other bodily discharge. However, conventional menstrual underwear provides fluid paths that may enable fluid absorbed thereby to leak onto an outer layer of the underwear. Through applied effort, ingenuity, and innovation, many deficiencies of such prior reusable menstrual underwear have been solved by developing solutions that are structured in accordance with the embodiments of the present invention, many examples of which are described in detail herein.

### BRIEF SUMMARY

Various embodiments provide leak-resistant and/or leak-proof underwear. For example, various embodiments provide underwear configured to collect and contain fluid such as menstrual fluid, urine, sweat, and/or the like. In an example embodiment, the underwear includes a gusset having a sealed multi-layer construction. The sealed multi-layer construction includes a sealed gusset assembly and a gusset portion of a body fabric. The body fabric generally makes up and/or forms the body of the underwear. The sealed gusset assembly includes a next-to-skin layer, an absorbent layer, a blocking layer, sealing tape, and a polymer component. The absorbent layer is disposed between the next-to-skin layer and the blocking layer and the sealing tape forms a seal between the top or bottom surface(s) of the next-to-skin layer and the top or bottom surface(s) of the blocking layer along a portion of respective perimeters of the next-to-skin layer and the blocking layer. The polymer component forms a seal between the top or bottom surface(s) of the next-to-skin layer and the top or bottom surface(s) of the block layer along at least one lateral edge of the respective perimeters of the next-to-skin layer and the blocking layer. The sealed gusset assembly is configured to be disposed between a wearer's skin and the gusset portion of the body fabric when the wearer is wearing the underpants.

In various embodiments, the sealing tape and/or the polymer component is secured and/or adhered (e.g., not stitched) to the at least a portion of the respective perimeters of the next-to-skin layer and the blocking layer.

### BRIEF DESCRIPTION OF THE SEVERAL VIEWS OF THE DRAWING(S)

Having thus described the invention in general terms, reference will now be made to the accompanying drawings, which are not necessarily drawn to scale, and wherein:

FIG. 1 provides a schematic diagram of leak-resistant and/or leak-proof underwear, in accordance with an example embodiment;

FIG. 2 provides a top view of an example multi-layer construction of a gusset of the leak-resistant and/or leak-proof underpants, in accordance with an example embodiment;

FIG. 3 provides a cross-sectional view of the example multi-layer construction of the gusset illustrated in FIG. 2;

FIG. 4 provides an illustration of how the sealed gusset assembly collects and contains fluid and prevents the fluid from reaching the body fabric, in accordance with an example embodiment;

FIGS. 5A, 5B, 6A, 6B, 7, 8, and 9 each illustrate a cross-sectional view of further example multi-layer construction of a gusset, in accordance with various embodiments; and

FIG. 10 provides some non-limiting examples of cross-sectional shapes of the polymer component, in accordance with various embodiments.

### DETAILED DESCRIPTION

Various embodiments now will be described more fully hereinafter with reference to the accompanying drawings, in which some, but not all embodiments are shown. Indeed, various embodiments may have many different forms and should not be construed as limited to the embodiments set forth herein; rather, these embodiments are provided so that this disclosure will satisfy applicable legal requirements. Like numbers refer to like elements throughout.

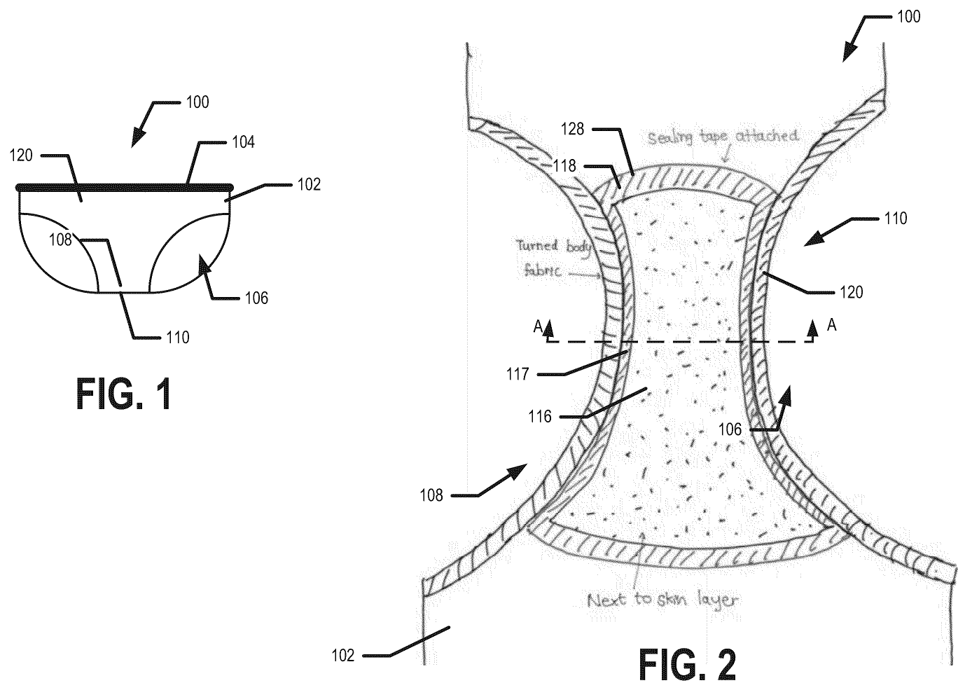

FIG. 1 illustrates an example pair of leak-resistant and/or leak-proof underwear 100. In various embodiments, a pair of leak-resistant and/or leak-proof underwear 100 includes an underwear body 102 primarily formed of a body fabric 120. The underwear body 102 has a waistband 104 secured thereto (e.g., via stitching, adhesive, securing, and/or the like) that is configured to fit and/or be positioned around a wearer's torso or hips when the wearer is wearing the underwear 100. The pair of leak-resistant and/or leak-proof underwear 100 further includes a gusset area 108 configured to be primarily positioned between a wearer's legs when the wearer is wearing the underwear 100. For example, the gusset area 108 is generally disposed between the leg holes 106.

The gusset area 108 includes multi-layer construction 110 configured to collect and contain fluid so as to prevent the fluid from reaching the body fabric 120. For example, fluid, such as menstrual fluid, urine, sweat, and/or the like, may be collected and contained by the multi-layer construction 110 so as to prevent the fluid from reaching the body fabric 120.

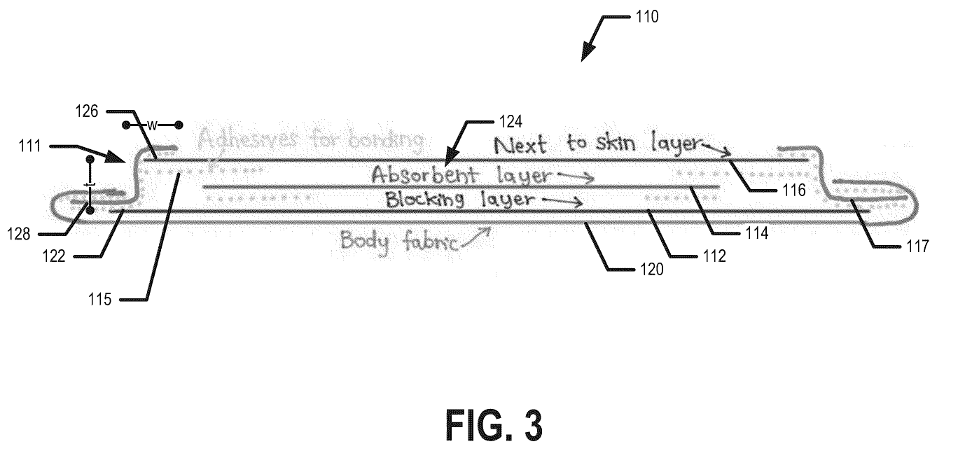

FIG. 2 provides a top view of the gusset area 108 of an example pair of leak-resistant and/or leak-proof underwear. FIG. 3 provides a cross-section view of the gusset area 108 taken along the AA line shown in FIG. 2.

In various embodiments, the multi-layer construction 110 includes a portion of the body fabric 120 disposed in the gusset area 108 of the underwear 100. In various embodiments, the multi-layer construction 110 further includes a sealed gusset assembly 111. In various embodiments, the sealed gusset assembly 111 includes a next-to-skin layer 116, an absorbent layer 114, and a blocking layer 112. The absorbent layer 114 is disposed between the next-to-skin layer 116 and the blocking layer 112.

The sealed gusset assembly 111 further comprises a polymer component 117 and sealing tape 118. The polymer component 117 and the sealing tape 118 form a seal between the top or bottom surface of the next-to-skin layer 116 and the top or bottom surface of the blocking layer 112 along the next-to-skin layer perimeter 126 and the blocking layer perimeter 122. For example, the polymer component 117 and the sealing tape 118 form a fluid-proof (e.g., waterproof) seal between the top or bottom surface of the next-to-skin layer 116 and the top or bottom surface of the blocking layer 112 along the next-to-skin layer perimeter 126 and the blocking layer perimeter 122. In various embodiments, the polymer component 117 forms the seal between the top or bottom surface of the next-to-skin layer 116 and the top or bottom surface of the blocking layer 112 along at least one lateral edge of the next-to-skin layer perimeter 126 and the blocking layer perimeter 122 (e.g., along at least a portion of a perimeter of at least one leg hole 106).

In various embodiments, the sealing tape 118 is coupled and/or sealed to the next-to-skin layer perimeter 126 and the blocking layer perimeter 122 via adhesive 115 or bonding. For example, the sealing tape 118 is secured to the next-to-skin layer perimeter 126 and the blocking layer perimeter 122 without the use of stitching. For example, the sealing tape 118 may be configured such that a tacking of the sealing tape 118 to the yarn structure of the next-to-skin layer 116, blocking layer 112, and/or body fabric 120 by means of mechanical forces, for example.

In various embodiments, the polymer component 117 is extruded directly onto the perimeter of the gusset area 108. For example, additional bonding or securing of the polymer component to the gusset area is not required in, various embodiments. For example, the molding pressure used in the molding and/or extruding of the polymer component 117 prevents and/or minimizes air gaps between the component and the gusset assembly. In various embodiments, the high pressure within the molten polymer is molded into the polymer component 117 causes the molten polymer to be fed into the fiber structure of the next-to-skin layer 116 and/or the block layer 112 so as to form a mechanical link between the resulting polymer component 117 and the next-to-skin layer 116 and/or the block layer 112.

In various embodiments, the next-to-skin layer 116, blocking layer 112, and the polymer component 117 and scaling tape 118 that couples the blocking layer perimeter 122 to the next-to-skin layer perimeter 126 define a fluid containment enclosure 124. The next-to-skin layer 116, blocking layer 112, polymer component 117, sealing tape 118, and adhesive 115 used to secure the sealing tape 118 to the blocking layer perimeter 122 and the next-to-skin layer perimeter 126 are configured to permit fluid to enter the fluid containment enclosure 124 via the next-to-skin layer 116 and then to maintain the fluid within the fluid containment enclosure 124. For example, the next-to-skin layer 116, blocking layer 112, polymer component 117, scaling tape 118, securing of the polymer component 117/sealing tape 118 to the blocking layer perimeter 122 and securing of the polymer component 117/sealing tape 118 to the next-to-skin layer perimeter 126 are configured to seal fluid within the fluid containment enclosure 124.

In various embodiments, the next-to-skin layer 116 is configured to be adjacent and/or in contact with a wearer's skin when the wearer is wearing the leak-resistant and/or leak-proof underwear 100. The next-to-skin layer 116 is formed of a material having one-way wicking properties. For example, the next-to-skin layer 116 is a one-direction wicking fabric. For example, the next-to-skin layer 116 is configured to wick fluid toward the absorbent layer 114. For example, the next-to-skin layer 116 is configured to wick fluid into the fluid containment enclosure 124 and to prevent wicking of fluid out of the fluid containment enclosure 124. For example, the next-to-skin layer 116 is configured to act as a one-way membrane, in various embodiments. In various embodiments, the next-to-skin layer 116 is a polyester-based fabric or a cellulosic fabric. In various embodiments, the next-to-skin layer 116 is formed of a material having anti-microbial properties.

In various embodiments, the absorbent layer 114 is configured to absorb and contain or hold fluid. In an example embodiment, the absorbent layer 114 is formed of a fluid absorbing composite material. In an example embodiment, the absorbent layer 114 is formed of a cellulose-based material, polyester-based material, a cotton-based material, a modal viscose seaweed-based material, and/or the like. In various embodiments, the material of the absorbent layer is warp knitted, weft knitted, woven, non-woven, of spacer construction, and/or the like. In an example embodiment, the absorbent layer 114 is a fluid absorbing foam, such as polyvinyl alcohol (PVA) and/or the like.

In various embodiments, the blocking layer 112 is a hydrophobic layer configured to contain fluid within the fluid containment enclosure 124 such that the fluid does not reach the body fabric 120. For example, the blocking layer 112 may be a hydrophobic membrane. For example, in various embodiments, the blocking layer 112 is formed of a polyester-based material. In an example embodiment, the blocking layer 112 is formed of a polyester-based material bonded to and/or including a waterproof and/or hydrophobic film. For example, the blocking layer 112 comprises a polyester-based material bonded to a polyurethane (PU) film, in an example embodiment.

In various embodiments, the sealing tape 118 is configured to seal one or more edges of the fluid containment enclosure 124. For example, in various embodiments, the sealing tape is formed of a hydrophobic material. For example, the sealing tape 118 is formed of a polyester-based material, in an example embodiment. For example, the sealing tape 118 is formed of a polyester-based material bonded to and/or including a waterproof and/or hydrophobic film, in an example embodiment.

In an example embodiment, the polymer component 117 is a hydrophobic extrusion and/or a compression molded component. In some embodiments, the polymer component 117 is formed of a hydrophobic thermoplastic or thermoset polymer. For example, in various scenarios, the polymer component 117 comprises silicone, polyurethane, acrylonitrile butadiene styrene (ABS), polycarbonate, epoxy, or another thermoplastic polymer or thermoset polymer that is hydrophobic. In various embodiments, the polymer component 117 is a Thermoplastic polyurethane (TPU) or a two-component curing system.

In an example embodiment, the polymer component 117 is formed on the gusset edge and/or perimeter via compression molding. For example, the polymer component 117 may be molded and/or extruded onto the blocking layer perimeter 122 and the next-to-skin layer perimeter 126. In various embodiments, molten or liquid polymer used to form the polymer component is fed to a compression cavity by manual means or through an extruder. The compression cavity is then used to form the polymer component 117 along at least a portion of the perimeter or edge of the gusset area 108 (e.g., along at least a portion of the perimeter of at least one leg hole 106).

In some embodiments, the polymer component 117 is configured to seal the entire perimeter of the gusset area (e.g., seal around the entirety of the blocking layer perimeter 122 and the next-to-skin layer perimeter 126). For example, some embodiments do not include scaling tape 118.

In various embodiments, a portion of the polymer component 117 and a portion of the sealing tape 118 that is secured to the next-to-skin layer perimeter 126 has a width w. In various embodiments, the width w is any (non-zero) value less than 20 mm (e.g., in a range of 2 to 15 mm, in an example embodiment). In an example embodiment, at least a (non-zero) portion of this width w (e.g., up to 20 mm) is exposed and/or visible from the top of the multi-layer construction 110, as shown in FIG. 2.

In various embodiments, a portion of the polymer component 117 and a portion of the sealing tape 118 that is secured to the blocking layer perimeter 122 has a width that is any (non-zero) value less than 20 mm (e.g., in a range of 2 to 15 mm, in an example embodiment). In various embodiments, a tail 128 of the polymer component 117 and/or sealing tape 118 extends outward from the portion of the polymer component 117 and/or sealing tape 118 that is secured to the blocking layer perimeter 122. In various embodiments, the body fabric 120 is secured to the tail 128 of the polymer component 117 and/or sealing tape 118 via adhesive 115 or securing technique. As shown in FIG. 3, in various embodiments, the body fabric 120 is turned onto and secured to the polymer component 117 and/or sealing tape in the hem area along the leg holes 106. In various embodiments, a tape (e.g., with a width of 1-20 mm) is made out of the body fabric and attached along the leg holes. At the edges of the gusset area 108 that are not along the leg holes 106, tail 128 of the polymer component 117 and/or sealing tape 118 may be left exposed.

In various embodiments, the seal along the perimeter of the sealed gusset assembly 111 has a thickness t. The thickness t is at least as thick as the absorbent layer 114. For example, in various embodiments, the thickness t is in a range of the thickness of the absorbent layer 114 to 8 mm thicker than the thickness of the absorbent layer. In an example embodiment, the polymer component 117 and/or sealing tape 118 has a thickness that is greater than 0 mm and may be up to 5 mm.

In various embodiments, the layers of the sealed gusset assembly 111 are adhered and/or secured to one another. For example, at least a portion of the next-to-skin layer 116 may be adhered and/or secured (e.g., via adhesive 115) to the absorbent layer 114 and/or blocking layer 112. In another example, at least a portion of the absorbent layer 114 is adhered and/or secured (e.g., via adhesive 115) to the blocking layer 112.

In various embodiments, the blocking layer 112 is wider than the next-to-skin layer 116. In various embodiments, the blocking layer 112 and the next-to-skin layer 116 are both wider than the absorbent layer 114.

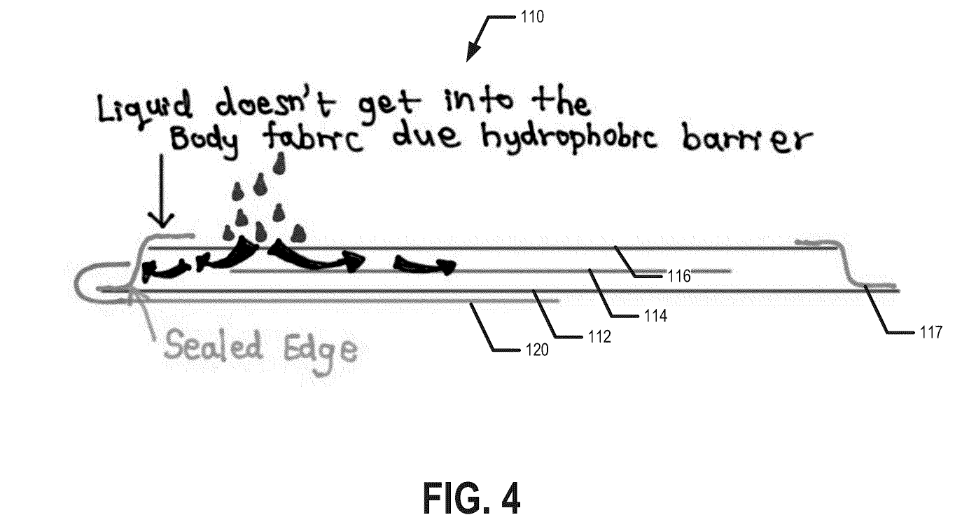

FIG. 4 provides an illustration of the containment of fluid within the fluid containment enclosure 124 by the sealed gusset assembly 111 such that the fluid is not able to reach the body fabric 120. For example, the fluid is incident on the next-to-skin layer 116 and is wicked into the fluid containment enclosure 124 through the next-to-skin layer 116. Within the fluid containment enclosure 124, at least a portion of the fluid is absorbed by the absorbent layer 114. As the fluid is absorbed into the absorbent layer 114, the fluid may be dispersed throughout the fluid containment enclosure 124. However, due to the sealing of the perimeter of the fluid containment enclosure 124 by the polymer component 117 and the sealing tape 118 being secured to the respective next-to-skin layer perimeter 126 and blocking layer perimeter 122, the fluid is not able to exit the fluid containment enclosure 124 via the perimeter thereof. For example, the polymer component 117 and the sealing tape 118 prevents the fluid within the fluid containment enclosure 124 from exiting the perimeter of the fluid containment enclosure 124 and reaching the body fabric 120.

In various embodiments, the leak-resistant and/or leak-proof underwear 100 is reusable underwear. For example, the leak-resistant and/or leak-proof underwear 100 is configured to be laundered. For example, in various embodiments, the blocking layer 112, polymer component 117, sealing tape 118, and/or next-to-skin layer 116 are configured to contain fluid within the fluid containment enclosure 124 and, in an example embodiment, to permit vapor (e.g., vaporized fluid) to exit the fluid containment enclosure 124. In an example embodiment, the blocking layer 112, polymer component 117, sealing tape 118, and/or next-to-skin layer 116 are configured to not permit vapor (e.g., vaporized fluid) to exit the fluid containment enclosure 124.

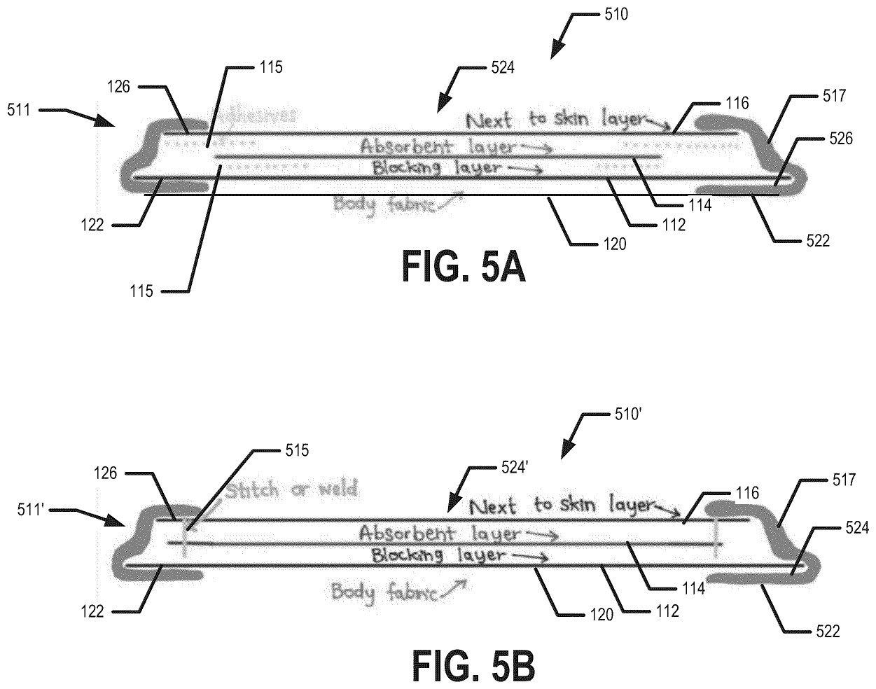

FIGS. 5A and 5B provide cross-section views of further embodiments of the gusset area 108 taken along the AA line shown in FIG. 2.

In various embodiments, the multi-layer construction 510, 510′ includes a portion of the body fabric 120 disposed in the gusset area 108 of the underwear 100. In various embodiments, the multi-layer construction 510, 510′ further includes a sealed gusset assembly 511, 511′. In various embodiments, the sealed gusset assembly 511, 511′ includes a next-to-skin layer 116, an absorbent layer 114, and a blocking layer 112. The absorbent layer 114 is disposed between the next-to-skin layer 116 and the blocking layer 112.

The sealed gusset assembly 511, 511′ further comprises polymer component 517 and, possibly sealing tape along a portion of the perimeter not shown. The polymer component 517 and any sealing tape form a seal between the top surface of the next-to-skin layer 116 along at least a portion of the next-to-skin layer perimeter 126 and the bottom surface of the blocking layer 112 along at least a portion of the blocking layer perimeter 122 and/or the body fabric 120. For example, the polymer component 517 forms a fluid-proof (e.g., waterproof) seal between the top surface of the next-to-skin layer 116 and the bottom surface of the blocking layer 112 along at least a portion of the next-to-skin layer perimeter 126 and the blocking layer perimeter 122. In various embodiments, the polymer component 517 is secured, coupled, and/or sealed to the next-to-skin layer perimeter 126 and the blocking layer perimeter 122. For example, the polymer component 517 may be compression molded onto the next-to-skin layer perimeter 126 and the blocking layer perimeter 122 without the use of stitching.

In various embodiments, the next-to-skin layer 116, blocking layer 112, polymer component 517, and any sealing tape that couples the blocking layer perimeter 122 to the next-to-skin layer perimeter 126 define a fluid containment enclosure 524, 524′. The next-to-skin layer 116, blocking layer 112, polymer component 517, any sealing tape, and any securing agent used to secure the sealing tape to the blocking layer perimeter 122 and the next-to-skin layer perimeter 126 are configured to permit fluid to enter the fluid containment enclosure 524, 524′ via the next-to-skin layer 116 and then to maintain the fluid within the fluid containment enclosure 524, 524′. For example, the next-to-skin layer 116, blocking layer 112, polymer component 517, any sealing tape, securing of the sealing tape to the blocking layer perimeter 122 and securing of the sealing tape to the next-to-skin layer perimeter 126 are configured to seal fluid within the fluid containment enclosure 524, 524′.

In various embodiments, internal layers of the fluid containment enclosure 524 are secured to one another via adhesive 115. In some embodiments, internal layers of the fluid containment enclosure 524′ are secured to one another via stitching or welding 515. However, as any such stitching 515 is internal to the fluid containment enclosure 524′, the stitching 515 is not present on any seam or boundary configured to contain and/or prevent passage of fluid. For example, any stitching 515 that passes through the next-to-skin layer 116 or the blocking layer 112 may be covered by the polymer component 517 or sealing tape such that the stitching 515 does not provide a path for fluid to exit the fluid containment enclosure 524′.

In various embodiments, the polymer component 517 and any sealing tape present is configured to seal one or more edges of the fluid containment enclosure 524, 524′. In the example multi-layer constructions 510, 510′, the polymer component 517 is molded to form a generally C-shaped seal (or backward C-shaped seal) so as to enclose and/or seal the edges of the multi-layer construction 510, 510′. For example, the polymer component 517 (in the cross-section view shown in FIGS. 5A and 5B) may be generally C-shaped (or backward C-shaped) so as to seal one or more edges of the multi-layer construction 510, 510′ through the securing of the polymer component 517 to a top surface of the next-to-skin layer 116 and a bottom surface of the blocking layer 112.

In the example multi-layer constructions 510, 510′, the body fabric 120 is not folded around the edge of the polymer component 517. Rather, an edge of the body fabric 120 may be secured and/or otherwise secured to the bottom surface 522 of the polymer component 517 or may be secured and/or otherwise secured to an internal surface 526 of the polymer component 517. In an example embodiment, the blocking layer 112 is the body fabric 120 of the gusset 108.

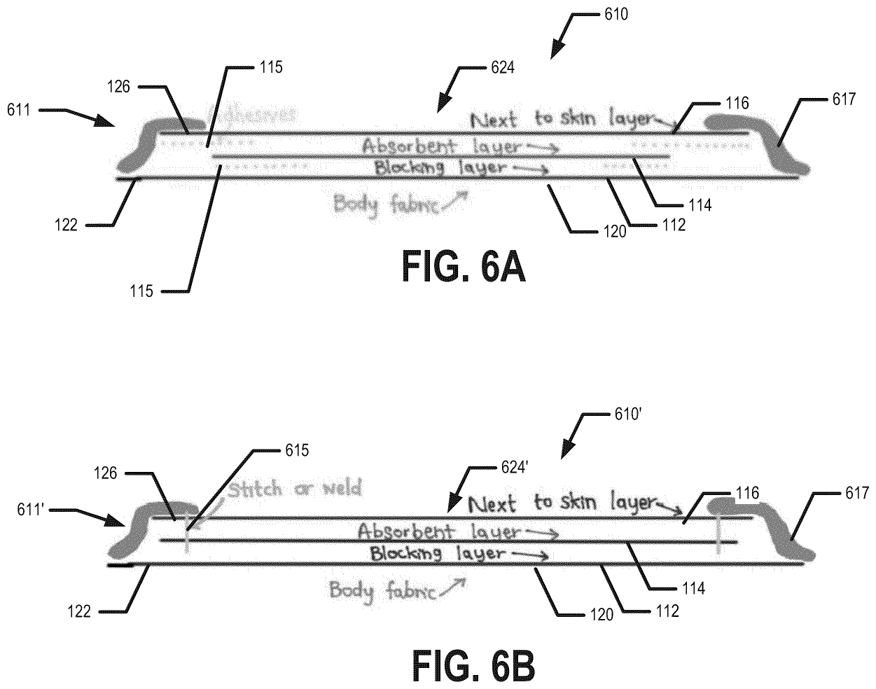

FIGS. 6A and 6B provide cross-section views of further embodiments of the gusset area 108 taken along the AA line shown in FIG. 2.

In various embodiments, the multi-layer construction 610, 610′ includes a portion of the body fabric 120 disposed in the gusset area 108 of the underwear 100. In various embodiments, the multi-layer construction 610, 610′ further includes a sealed gusset assembly 611, 611′. In various embodiments, the sealed gusset assembly 611, 611′ includes a next-to-skin layer 116, an absorbent layer 114, and a blocking layer 112. The absorbent layer 114 is disposed between the next-to-skin layer 116 and the blocking layer 112.

The sealed gusset assembly 611, 611′ further comprises a polymer component 617 and may include sealing tape along a portion of the perimeter of the gusset area 108 that is not shown. The polymer component 617 and any sealing tape present form a seal between the top surface of the next-to-skin layer 116 along at least a portion of the next-to-skin layer perimeter 126 and the top surface of the blocking layer 112 along at least a portion of the blocking layer perimeter 122 and/or the body fabric 120. For example, the polymer component 617 and any sealing tape present form a fluid-proof (e.g., waterproof) seal between the top surface of the next-to-skin layer 116 and the top surface of the blocking layer 112 along at least a portion of the next-to-skin layer perimeter 126 and the blocking layer perimeter 122. In various embodiments, the polymer component 617 and any sealing tape present is secured, molded onto, coupled and/or sealed to the next-to-skin layer perimeter 126 and the blocking layer perimeter 122. For example, any sealing tape may be secured (e.g., via heat securing and/or welding) to the next-to-skin layer perimeter 126 and the blocking layer perimeter 122 without the use of stitching. In another example, the polymer component 617 may be compression molded onto the next-to-skin layer perimeter 126 and the blocking layer perimeter 122 (e.g., without the use of stitching).

In various embodiments, the next-to-skin layer 116, blocking layer 112, and the polymer component 617 and the sealing tape that couples the blocking layer perimeter 122 to the next-to-skin layer perimeter 126 define a fluid containment enclosure 624, 624′. The next-to-skin layer 116, blocking layer 112, polymer component 617, scaling tape, and any securing agent used to secure the sealing tape to the blocking layer perimeter 122 and the next-to-skin layer perimeter 126 are configured to permit fluid to enter the fluid containment enclosure 624, 624′ via the next-to-skin layer 116 and then to maintain the fluid within the fluid containment enclosure 624, 624′. For example, the next-to-skin layer 116, blocking layer 112, polymer component 617, sealing tape, securing of the polymer component 617 and sealing tape to the blocking layer perimeter 122 and securing of the polymer component 617 and sealing tape to the next-to-skin layer perimeter 126 are configured to seal fluid within the fluid containment enclosure 624, 624′.

In various embodiments, internal layers of the fluid containment enclosure 624 are secured to one another via adhesive 115. In some embodiments, internal layers of the fluid containment enclosure 624′ are secured to one another via stitching or welding 615. However, as any such stitching 615 is internal to the fluid containment enclosure 624′, the stitching 615 is not present on any seam or boundary configured to contain and/or prevent passage of fluid. For example, any stitching 615 that passes through the next-to-skin layer 116 or the blocking layer 112 may be covered by the polymer component 617 or sealing tape such that the stitching 615 does not provide a path for fluid to exit the fluid containment enclosure 624′.

In various embodiments, the polymer component 617 and, optionally, sealing tape are configured to seal one or more edges of the fluid containment enclosure 624, 624′. In the example multi-layer constructions 610, 610′, the polymer component 617 is molded to form a generally S-shaped seal (or backward S-shaped seal) so as to enclose and/or seal the edges of the multi-layer construction 610, 610′. For example, the polymer component 617 (in the cross-section view shown in FIGS. 6A and 6B) may be generally S-shaped (or backward (S-shaped) so as to seal one or more edges of the multi-layer construction 610, 610′ through the securing of the polymer component 617 to a top surface of the next-to-skin layer 116 and a top surface of the blocking layer 112.

In the example multi-layer constructions 610, 610′, the body fabric 120 is not folded around the edge of the polymer component 617. Rather, an edge of the body fabric 120 may be secured and/or otherwise secured to the bottom surface of the blocking layer 112. In an example embodiment, the blocking layer 112 is the body fabric 120 of the gusset 108.

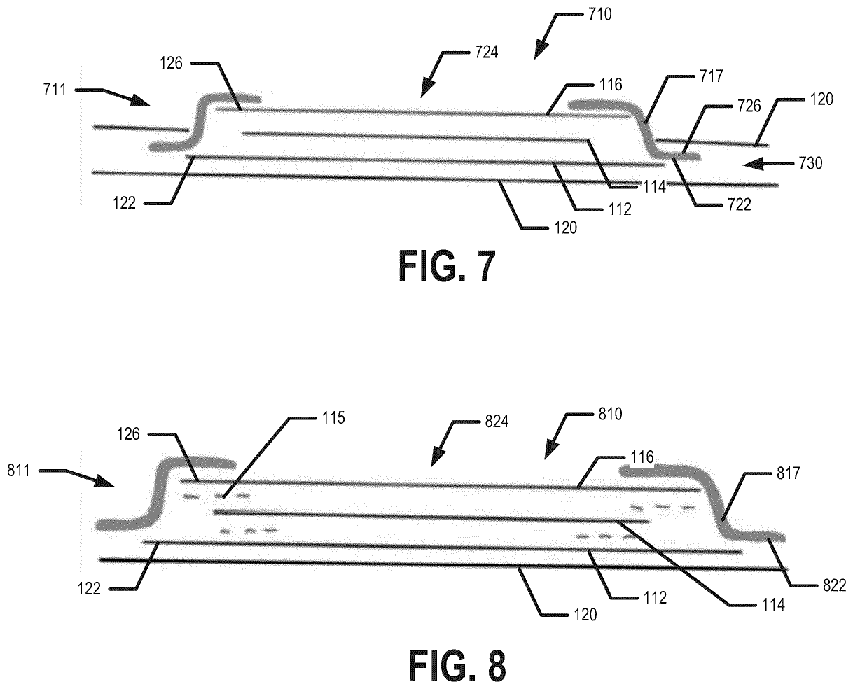

FIG. 7 provides a cross-section view of another embodiment of the gusset area 108 taken along the AA line shown in FIG. 2.

In various embodiments, the multi-layer construction 710 includes a portion of the body fabric 120 disposed in the gusset area 108 of the underwear 100. In various embodiments, the multi-layer construction 710 further includes a sealed gusset assembly 711. In various embodiments, the sealed gusset assembly 711 includes a next-to-skin layer 116, an absorbent layer 114, and a blocking layer 112. The absorbent layer 114 is disposed between the next-to-skin layer 116 and the blocking layer 112.

The sealed gusset assembly 711 further comprises polymer component 717. The polymer component 717 forms a seal between the top surface of the next-to-skin layer 116 along at least a portion of the next-to-skin layer perimeter 126 and the top surface of the blocking layer 112 along at least a portion of the blocking layer perimeter 122 and/or the body fabric 120. In some embodiments, the sealed gusset assembly 711 further comprises sealing tape configured to seal a portion of the perimeter of the gusset area 108 not shown. For example, the polymer component 717 and any sealing tape present form a fluid-proof (e.g., waterproof) seal between the top surface of the next-to-skin layer 116 and the top surface of the blocking layer 112 along at least a portion of the next-to-skin layer perimeter 126 and the blocking layer perimeter 122. In various embodiments, the polymer component 717, and any sealing tape present, is coupled and/or sealed to the next-to-skin layer perimeter 126 and the blocking layer perimeter 122. For example, the sealing tape 718 is secured (e.g., via heat bonding and/or welding) to the next-to-skin layer perimeter 126 and the blocking layer perimeter 122 without the use of stitching. For example, the polymer component 717 may be compression molded onto the next-to-skin layer perimeter 126 and the blocking layer perimeter 122 (e.g., without the use of stitching).

In various embodiments, the next-to-skin layer 116, blocking layer 112, and the polymer component 717 and any sealing tape that couples the blocking layer perimeter 122 to the next-to-skin layer perimeter 126 define a fluid containment enclosure 724. The next-to-skin layer 116, blocking layer 112, polymer component 717, any sealing tape, and any securing agent used to secure the sealing tape to the blocking layer perimeter 122 and the next-to-skin layer perimeter 126 are configured to permit fluid to enter the fluid containment enclosure 724 via the next-to-skin layer 116 and then to maintain the fluid within the fluid containment enclosure 724. For example, the next-to-skin layer 116, blocking layer 112, polymer component 717, any sealing tape, securing of the sealing tape to the blocking layer perimeter 122 and securing of the sealing tape to the next-to-skin layer perimeter 126 are configured to seal fluid within the fluid containment enclosure 724.

In various embodiments, internal layers of the fluid containment enclosure 724 are secured to one another via adhesive, stitching, welding, and/or the like. However, as any such stitching is internal to the fluid containment enclosure 724, the stitching is not present on any scam or boundary configured to contain and/or prevent passage of fluid. For example, any stitching that passes through the next-to-skin layer 116 or the blocking layer 112 may be covered by the polymer component 717 or sealing tape such that the stitching does not provide a path for fluid to exit the fluid containment enclosure 724.

In various embodiments, the polymer component 717 and any sealing tape present are configured to seal one or more edges of the fluid containment enclosure 724. In the example multi-layer construction 710, the polymer component 717 is generally S-shaped (or backward S-shaped) so as to enclose and/or seal the edges of the multi-layer construction 710. For example, the polymer component 717 (in the cross-section view shown in FIG. 7) may be generally S-shaped (or backward (S-shaped) so as to seal one or more edges of the multi-layer construction 710 through the securing of the polymer component 717 to a top surface of the next-to-skin layer 116 and a top surface of the blocking layer 112.

In the example multi-layer construction 710, the polymer component 717 may also be secured to a portion of the body fabric 120. For example, a portion of the body fabric 120 may be secured to a bottom surface 722 of the polymer component 717. Another portion of the body fabric 120 may be secured to a top surface 726 of the polymer component 717. In an example embodiment, the portion of the body fabric 120 that is secured to the top surface 726 is a continuation of the portion of the body fabric 120 secured to the bottom surface 722 of the polymer component 717 that has been folded over. In another example embodiment, the portion of the body fabric 120 that is secured to the top surface 726 is a different piece of body fabric than the portion of the body fabric 120 secured to the bottom surface 722 of the polymer component 717. For example, two different pieces of body fabric 120 may be secured and/or stitched together such that a pocket 730 is formed therebetween. The polymer component 717 may be secured to surfaces of the pocket 730.

FIG. 8 provides a cross-section view of another embodiment of the gusset area 108 taken along the AA line shown in FIG. 2.

In various embodiments, the multi-layer construction 810 includes a portion of the body fabric 120 disposed in the gusset area 108 of the underwear 100. In various embodiments, the multi-layer construction 810 further includes a sealed gusset assembly 811. In various embodiments, the sealed gusset assembly 811 includes a next-to-skin layer 116, an absorbent layer 114, and a blocking layer 112. The absorbent layer 114 is disposed between the next-to-skin layer 116 and the blocking layer 112.

The sealed gusset assembly 811 further comprises polymer component 817 and possibly sealing tape along a portion of the perimeter of the sealed gusset assembly 811 that is not shown. The polymer component 817 and any sealing tape present form a seal between the top surface of the next-to-skin layer 116 along at least a portion of the next-to-skin layer perimeter 126 and the top surface of the blocking layer 112 along at least a portion of the blocking layer perimeter 122 and/or the body fabric 120. For example, the polymer component 817 and any sealing tape form a fluid-proof (e.g., waterproof) seal between the top surface of the next-to-skin layer 116 and the top surface of the blocking layer 112 along at least a portion of the next-to-skin layer perimeter 126 and the blocking layer perimeter 122. In various embodiments, the polymer component 817 and any sealing tape present are coupled and/or sealed to the next-to-skin layer perimeter 126 and the blocking layer perimeter 122. For example, the sealing tape 818 is secured (e.g., via heat bonding and/or welding) to the next-to-skin layer perimeter 126 and the blocking layer perimeter 122 without the use of stitching. For example, the polymer component 817 may be compression molded onto the next-to-skin layer perimeter 126 and the blocking layer perimeter 122 (e.g., without the use of stitching).

In various embodiments, the next-to-skin layer 116, blocking layer 112, and the polymer component 817 and any scaling tape that couples the blocking layer perimeter 122 to the next-to-skin layer perimeter 126 define a fluid containment enclosure 824. The next-to-skin layer 116, blocking layer 112, polymer component 817, any sealing tape, and any securing agent used to secure the sealing tape to the blocking layer perimeter 122 and the next-to-skin layer perimeter 126 are configured to permit fluid to enter the fluid containment enclosure 824 via the next-to-skin layer 116 and then to maintain the fluid within the fluid containment enclosure 824. For example, the next-to-skin layer 116, blocking layer 112, polymer component 817, any scaling tape, securing of the sealing tape to the blocking layer perimeter 122 and securing of the sealing tape 818 to the next-to-skin layer perimeter 126 are configured to seal fluid within the fluid containment enclosure 824.

In various embodiments, internal layers of the fluid containment enclosure 824 are secured to one another via adhesive 115, stitching, welding, and/or the like. However, as any such stitching is internal to the fluid containment enclosure 824, the stitching is not present on any seam or boundary configured to contain and/or prevent passage of fluid. For example, any stitching that passes through the next-to-skin layer 116 or the blocking layer 112 may be covered by the polymer component 817 or sealing tape such that the stitching does not provide a path for fluid to exit the fluid containment enclosure 824.

In various embodiments, the polymer component 817 and any scaling tape is configured to seal one or more edges of the fluid containment enclosure 824. In the example multi-layer construction 810, the polymer component 817 is molded to form a generally S-shaped seal (or backward S-shaped seal) so as to enclose and/or seal the edges of the multi-layer construction 810. For example, the polymer component 817 (in the cross-section view shown in FIG. 8) may be generally S-shaped (or backward (S-shaped) so as to seal one or more edges of the multi-layer construction 810 through the securing of the polymer component 817 to a top surface of the next-to-skin layer 116 and a top surface of the blocking layer 112.

In the example multi-layer construction 810, the polymer component 817 may also be secured to a portion of the body fabric 120. For example, a portion of the body fabric 120 may be secured to a bottom surface 822 of the polymer component 817.

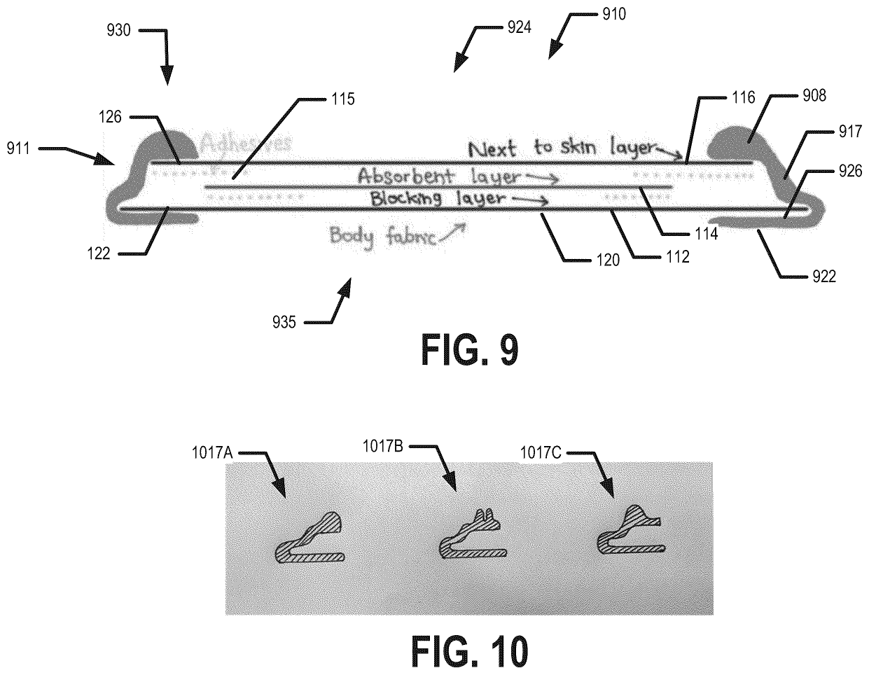

FIG. 9 provides a cross-section view of another embodiment of the gusset area 108 taken along the AA line shown in FIG. 2.

In various embodiments, the multi-layer construction 910 includes a portion of the body fabric 120 disposed in the gusset area 108 of the underwear 100. In various embodiments, the multi-layer construction 910 further includes a sealed gusset assembly 911. In various embodiments, the sealed gusset assembly 911 includes a next-to-skin layer 116, an absorbent layer 114, and a blocking layer 112. The absorbent layer 114 is disposed between the next-to-skin layer 116 and the blocking layer 112.

The sealed gusset assembly 911 further comprises a polymer component 917, and possibly sealing tape along a portion of the perimeter of the sealed gusset assembly 911 not shown. The polymer component 917, and any sealing tape present form a seal between the top surface of the next-to-skin layer 116 along at least a portion of the next-to-skin layer perimeter 126 and the bottom surface of the blocking layer 112 along at least a portion of the blocking layer perimeter 122 and/or the body fabric 120. For example, the polymer component 917, and any sealing tape present form a fluid-proof (e.g., waterproof) seal between the top surface of the next-to-skin layer 116 and the bottom surface of the blocking layer 112 along at least a portion of the next-to-skin layer perimeter 126 and the blocking layer perimeter 122. In various embodiments, the polymer component 917 and any sealing tape are coupled and/or sealed to the next-to-skin layer perimeter 126 and the blocking layer perimeter 122. For example, the scaling tape is secured (e.g., via heat bonding and/or welding) to the next-to-skin layer perimeter 126 and the blocking layer perimeter 122 without the use of stitching. For example, the polymer component 917 is compression molded onto the next-to-skin layer perimeter 126 and the blocking layer perimeter 122 (e.g., without the use of stitching).

In various embodiments, the next-to-skin layer 116, blocking layer 112, and the polymer component 917, and any sealing tape, that couples the blocking layer perimeter 122 to the next-to-skin layer perimeter 126 define a fluid containment enclosure 924. The next-to-skin layer 116, blocking layer 112, polymer component 917, any sealing tape, and any securing agent used to secure the sealing tape to the blocking layer perimeter 122 and the next-to-skin layer perimeter 126 are configured to permit fluid to enter the fluid containment enclosure 924 via the next-to-skin layer 116 and then to maintain the fluid within the fluid containment enclosure 924. For example, the next-to-skin layer 116, blocking layer 112, polymer component 917, any sealing tape, securing of the sealing tape to the blocking layer perimeter 122 and securing of the sealing tape to the next-to-skin layer perimeter 126 are configured to seal fluid within the fluid containment enclosure 924.

In various embodiments, internal layers of the fluid containment enclosure 924 are secured to one another via adhesive 115. In some embodiments, internal layers of the fluid containment enclosure 924 are secured to one another via stitching or welding. However, as any such stitching is internal to the fluid containment enclosure 924. For example, the stitching is not present on any seam or boundary configured to contain and/or prevent passage of fluid. For example, any stitching that passes through the next-to-skin layer 116 or the blocking layer 112 may be covered by the polymer component 917 or sealing tape such that the stitching does not provide a path for fluid to exit the fluid containment enclosure 924.

In various embodiments, the polymer component 917 and any sealing tape present are configured to seal one or more edges of the fluid containment enclosure 924. In the example multi-layer construction 910, the polymer component 917 is molded to form a generally C-shaped seal (or backward C-shaped seal) so as to enclose and/or seal the edges of the multi-layer construction 910. For example, the polymer component 917 (in the cross-section view shown in FIG. 9) may be generally C-shaped (or backward C-shaped) so as to seal one or more edges of the multi-layer construction 910 through the securing of the polymer component 917 to a top surface of the next-to-skin layer 116 and a bottom surface of the blocking layer 112.

In other embodiments, the polymer component 917 (in the cross-section view shown in FIG. 9) may be generally S-shaped (or backward S-shaped) so as to seal one or more edges of the multi-layer construction 910 through the securing of the polymer component 917 to a top surface of the next-to-skin layer 116 and a top surface of the blocking layer 112.

In an example multi-layer construction 910, the body fabric 120 is folded around the edge of the polymer component 917. In another example multi-layer construction 910, an edge of the body fabric 120 may be secured and/or otherwise secured to the bottom surface 922 of the polymer component 917 or may be secured and/or otherwise secured to an internal surface 926 of the polymer component 917. In an example embodiment, the blocking layer 112 is the body fabric 120 of the gusset 108.

In various embodiments, the polymer component 917 includes a bump or ridge 908 along at least a portion of the next-to-skin layer perimeter 126. For example, the sealing tape 918 may be shaped (e.g., extruded and/or molded) to form three-dimensional (3D) structured lines along the edges (e.g., leg seams) of the gusset 108. For example, the polymer component 917 may include a thicker portion thereof that forms a bump or ridge 908. For example, the edge 930 of the multi-layer construction 910 may be thicker than a central portion 935 of the multi-layer construction 910.

FIG. 10 illustrates some non-limiting examples of cross-sectional shapes of polymer components 1017A, 1017B, 1017C, according to various embodiments. For example, the profile or cross-sectional shape of the polymer component can be any shape as per the mold cavity. In various embodiments, the profile or cross-sectional shape of the polymer component may be a raised profile that acts as an extra barrier for fluids. For example, the raised profile may extend outwardly from the next-to-skin layer. In various embodiments, the polymer component may have an inherent stickiness or tackiness which may give extra anchoring of the gusset to the skin of a wearer. In an example embodiment, the polymer component comprises a material that has antibacterial properties and/or has been impregnated with a material that has antibacterial properties.

Many modifications and other embodiments will come to mind to one skilled in the art to which this disclosure pertains having the benefit of the teachings presented in the foregoing descriptions and the associated drawings. Therefore, it is to be understood that the embodiments are not to be limited to the specific embodiments disclosed and that modifications and other embodiments are intended to be included within the scope of the appended claims. Although specific terms are employed herein, they are used in a generic and descriptive sense only and not for purposes of limitation.

1. Leak-resistant underwear comprising:
a gusset having a multi-layer construction, the multi-layer construction comprising a sealed gusset assembly and a gusset portion of a body fabric, the sealed gusset assembly comprising:
a next-to-skin layer,
an absorbent layer,
a blocking layer and
a polymer component
wherein the absorbent layer is disposed between the next-to-skin layer and the blocking layer and the polymer component forms a seal between a top or bottom surface of the next-to-skin layer and a top or bottom surface of the blocking layer, and
the sealed gusset assembly is configured to be disposed between a wearer's skin and the gusset portion of the body fabric.

2. The leak-resistant underwear of claim 1, further comprising sealing tape that forms a seal between the top or bottom surface of the next-to-skin layer and the top or bottom of the block layer along a portion of the respective perimeters of the next-to-skin layer and the blocking layer, wherein the sealing tape is adhered or secured to the portion of the respective perimeters of the next-to-skin layer and the blocking layer.

3. The leak-resistant underwear of claim 2, wherein at least 1 mm of the sealing tape and at least 1 mm of the polymer component is exposed between the next-to-skin layer and the body fabric.

4. The leak-resistant underwear of claim 2, wherein at least one of the blocking layer, the polymer component, or the sealing tape is configured to prevent fluid from passing therethrough and may or may not permit vapor to pass therethrough.

5. The leak-resistant underwear of claim 2, wherein the next-to-skin layer, blocking layer, polymer component, and sealing tape define a fluid containment enclosure and are configured to contain fluid therein.

6. The leak-resistant underwear of claim 5, wherein at least one of the next-to-skin layer, blocking layer, polymer component, or sealing tape may or may not permit vapor to exit the fluid containment enclosure.

7. The leak-resistant underwear of claim 1, wherein the polymer component extends along at least a portion of respective perimeters of the next-to-skin layer and the blocking layer.

8. The leak-resistant underwear of claim 1, wherein the next-to-skin layer is a one-direction wicking fabric configured to wick fluid toward the absorbent layer.

9. The leak-resistant underwear of claim 1, wherein the polymer component comprises at least one of a polymer material, a silicone material, or an epoxy material.

10. The leak-resistant underwear of claim 1, wherein the absorbent layer comprises at least one of a cellulose-based material, polyester-based material, a cotton-based material, a modal viscose seaweed-based material, or fluid absorbing foam.

11. The leak-resistant underwear of claim 1, wherein the blocking layer is formed of a hydrophobic material.

12. The leak-resistant underwear of claim 1, wherein the polymer component is formed of an extruded and/or compression molded hydrophobic material.

13. The leak-resistant underwear of claim 1, wherein the polymer component is secured to an edge of the body fabric.

14. The leak-resistant underwear of claim 1, wherein the next-to-skin layer is formed of a polyester-based fabric or cellulosic fiber.

15. The leak-resistant underwear of claim 1, wherein the blocking layer is formed of at least one of a polyester-based fabric including or secured to a waterproof film, silicone, polyurethane, acrylonitrile butadiene styrene (ABS), polycarbonate, or another thermoplastic polymer or thermoset polymer that is hydrophobic.

16. The leak-resistant underwear of claim 1, wherein the blocking layer is wider than the next-to-skin layer.

17. The leak-resistant underwear of claim 1, wherein both the blocking layer and the next-to-skin layer are wider than the absorbent layer.

18. The leak-resistant underwear of claim 1, wherein the polymer component is compression molded or extruded onto the at least a portion of respective perimeters of the next-to-skin layer and the blocking layer.

19. The leak-resistant underwear of claim 1, wherein the polymer component has a raised profile.

20. Leak-resistant underwear comprising:
a gusset having a multi-layer construction, the multi-layer construction comprising a sealed gusset assembly and a gusset portion of a body fabric, the sealed gusset assembly comprising:
a next-to-skin layer,
an absorbent layer,
a blocking layer,
sealing tape, and
a polymer component,
wherein the absorbent layer is disposed between the next-to-skin layer and the blocking layer and the polymer component and the sealing tape form a seal between respective portions of a top or bottom surface of the next-to-skin layer and a top or bottom surface of the blocking layer along respective perimeters of the next-to-skin layer and the blocking layer,
wherein the sealed gusset assembly is configured to be disposed between a wearer's skin and the gusset portion of the body fabric, and
wherein at least 1 mm of the sealing tape and at least 1 mm of the polymer component is exposed between the next-to-skin layer and the body fabric.
